(19) United States
(12) Patent Application Publication
(10) Pub. No.: US 2009/0155199 A1
(43) Pub. Date: Jun. 18, 2009

(54) Apparatus and methods for pain relief using ultrasound energized polymers

(76) Inventor: Eilaz Babaev, Minnetonka, MN (US)

Correspondence Address: Bacoustics, LLC, 5929 BAKER ROAD, SUITE 470, MINNETONKA, MN, 55345, US

(21) Appl. No.: 11/409,818

(22) Filed: Apr. 24, 2006

(51) Int. Cl. A61K 31/74; B01J 19/10; B06B 1/00

(52) U.S. Cl. 424/78.02; 204/157.62; 422/128

(57) ABSTRACT

Method and device to create energized polymers that can be used for pain relief, comprised of an ultrasound system that ultrasonically energize polymers that can then be used to provide an analgesic effect. Ultrasound waves are delivered to a polymer through direct contact, through a coupling medium, or without contact in order to energize the polymer. Other energies such as such as UV, microwave, laser, electricity, RF, sun, light, magnetic/electromagnetic, et can also be used to energize the polymer. The energized polymer can be immediately placed on a user to provide an analgesic effect, or the energized polymer can be placed storage material and removed at a later time to be placed on a user to provide an analgesic effect.

### BACKGROUND OF THE INVENTION

1. Field of the Invention

The present invention relates to pain relief. In particular, the present invention relates to apparatus and methods for pain relief using polymers energized by exposure to ultrasonic waves, and said polymers are capable of storing the energy imparted to them from ultrasound exposure.

2. Description of the Related Art

Treating persistent lingering pain, often but not exclusively associated with arthritis, muscles soreness, headache, etc, with various forms of energy is well known to the art. Most often the energy chosen is a variant of thermal energy, which in particular is heat or cold applied via a portable pad or pack. Applying thermal energy to a portable pack or pad is generally accomplished by means of a chemical reaction or energy transfer by placing the pad or pack in hot environment, such as boiling water or a microwave oven, or a cold environment, such as a fridge or freezer. Transferring thermal energy to a portable pad or pack often results in the pad or pack becoming overheated or overcooled. When placed on the user, an overheated pad or pack can cause the user discomfort or burn the user's skin. Similarly, an overcooled pad or pack when placed on the user's body can cause the user discomfort or freeze burn the user's skin.

Supplying thermal energy to a portable pad or pack can also be accomplished by placing two or more chemicals that are temporarily separated within the pack or pad—these chemicals can be combined to create an endothermic or exothermic chemical reaction. When the user is in need of pain relief, the user activates the pad or pack by removing the barrier separating the reactive chemicals. Though effective at producing thermal energy, the use of chemicals in portable packs or pads is hazardous in that the chemicals employed can injure the user's skin if the chemicals were to leak out of the pad or pack.

Imparting thermal energy to a location of persistent lingering pain is also accomplished by applying chemicals and creams to the affected area and allowing them to evaporate. Though not effective at generating heat, the evaporation of chemicals applied to the skin can generate a local cooling at the location of the user's body experiencing persistent lingering pain. The use of creams and chemicals is disadvantaged by the fact that such creams and chemicals are often messy to apply and can cause severe irritation if they come in contact with the user's eyes or mucosal membranes.

Generating and applying therapeutic energy to a location of the body experiencing persistent lingering pain is also accomplished by electrical stimulation. Transcutaneous Electrical Nerve Stimulation (TENS) is an example of this methodology. TENS, and other similar methods, treat pain by using electrodes to induce a current across the user's skin that transverses the site of persistent lingering pain. Portable versions of TENS, and similar devices, have been created and marketed. Requiring batteries or an external power source and often being bulking, TENS devices are not truly portable. Furthermore, the device is worthless if the user of the device is without batteries or an electrical outlet.

The limitations of the current energy based treatments of persistent lingering pain create a need for a portable device that is not bulky, that does not require the user to supply an external energy source or battery, that does not derive thermal energy from chemicals that irritate, injury, or burn the user's skin, and that cannot be overheated or overcooled as to avoid injuring the user.

### SUMMARY OF THE INVENTION

The present invention is directed towards apparatus and methods for pain relief by using polymers energized by exposure to ultrasound, and said polymers are capable of storing the energy imparted to them from ultrasound exposure. Apparatus and methods in accordance with the present invention may meet the above-mentioned needs and also provide additional advantages and improvements that will be recognized by those skilled in the art upon review of the present disclosure.

The present invention comprises an ultrasonic generator, an ultrasonic transducer, an ultrasound horn, and an ultrasound tip. Exposing a polymer to ultrasonic waves energizes the polymer and that polymer can then be used to provide pain relief.

Ultrasonic waves are delivered to a polymer in order to energize that polymer. Ultrasonic waves are delivered by directly contacting the polymer with the ultrasound tip, by contacting the polymer through a coupling medium, or without contacting the polymer. The energized polymer is applied to a user to provide an analgesic effect either immediately after being energized or the energized polymer can be stored for use at a future time.

The invention is related to apparatus and methods for pain relief that uses polymers energized by exposure to ultrasonic waves.

One aspect of this invention may be to provide a method and device for quick pain relief.

Another aspect of this invention may be to provide a method and device for more effective pain relief.

Another aspect of the invention may be to provide a method and device for more efficient pain relief.

Another aspect of the invention may be to provide a method and device for safer pain relief.

Another aspect of the invention may be to provide a method and device for pain relief that does not use chemicals or drugs.

Another aspect of this invention may be to provide a method and device for pain relief that is easy to use.

Another aspect of the invention may be to provide a method and device for pain relief that can be used at home by an individual.

Another aspect of the invention may be to provide a portable means for pain relief.

These and other aspects of the invention will become more apparent from the written descriptions and figures below.

### BRIEF DESCRIPTION OF THE DRAWINGS

The present Invention will be shown and described with reference to the drawings of preferred embodiments and clearly understood in details.

FIG. 1 is a perspective view for an ultrasound apparatus capable of energizing polymers according to the present invention.

FIG. 2 is a cross-sectional view of the ultrasound apparatus.

FIG. 3 are front-views of ultrasound tips that can be used with the ultrasound apparatus.

FIG. 4 is a perspective view of an ultrasound apparatus capable of energizing polymers through direct contact with a polymer.

FIG. 5 is a detailed view of an ultrasound apparatus that can energize polymers through direct contact with a polymer.

FIG. 6 is a perspective schematic view of a production line with an ultrasound apparatus capable of energizing polymers through direct contact.

FIG. 7 is a perspective schematic view of a production line with an ultrasound apparatus capable of energizing polymers through a coupling medium.

FIG. 8 is a perspective schematic view of an example production line with an ultrasound apparatus capable of energizing polymers and with a separate device to seal polymers in storage.

FIG. 9 is a perspective view of a production line with an ultrasound apparatus capable of both energizing polymers and sealing the energized polymers in storage.

FIG. 10 is a perspective view of a production line with a rotating ultrasound apparatus that can energize moving polymers from the radial side of an ultrasound tip.

FIG. 11 is a cross-sectional view of a production line with a rotating ultrasound tip capable of energizing moving polymers from the radial side of the ultrasound tip.

FIG. 12 is a cross-sectional view of a production line with an ultrasound tip in a fixed position that can energize moving polymers.

FIG. 13 is a cross-sectional view of a production line with two rotating ultrasound tips capable of energizing moving polymers from the radial side of ultrasound tips.

FIG. 14 is a cross-sectional view of a production line with a rotating ultrasound tip that is capable of energizing moving polymers from the radial side of the ultrasound tip.

### DETAILED DESCRIPTION OF THE INVENTION

The present invention is an apparatus and methods for pain relief using polymers energized by exposure to ultrasonic waves, and said polymers are capable of storing the energy imparted to them from ultrasound exposure. Preferred embodiments of the present invention in the context of an apparatus and methods are illustrated in the figures and described in detail below.

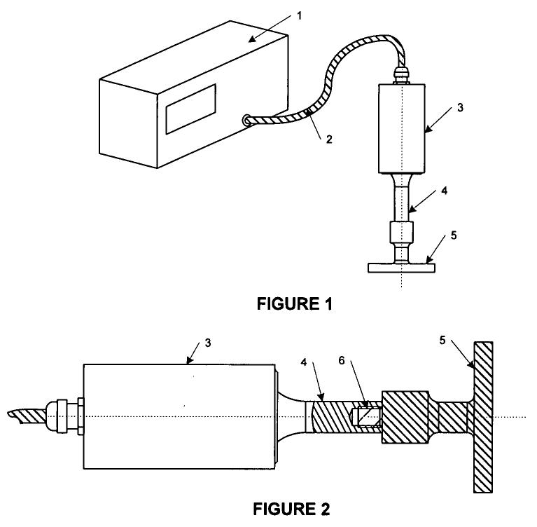

FIG. 1 is a perspective view for an ultrasound apparatus capable of energizing polymers according to the present invention. The ultrasound apparatus comprise an ultrasound power generator 1, a power supply cord 2, an ultrasonic transducer 3, an ultrasound horn 4, and an ultrasound tip 5.

FIG. 2 is a cross-sectional view of the ultrasound transducer 3 with accompanying ultrasound horn 4 and ultrasound tip 5 that is depicted in FIG. 1. The ultrasonic transducer 3 is connected to the ultrasound horn 4. The ultrasound horn 4 is mechanically connected to an ultrasound tip 5 by threading or other means 6. The preferred embodiment comprises an ultrasound tip 5 that is directly connected to the ultrasound horn 4 by a mechanical interface; alternative embodiments could have the ultrasound tip 5 directly connected to the ultrasound horn 4 to comprise a single piece without a mechanical interface.

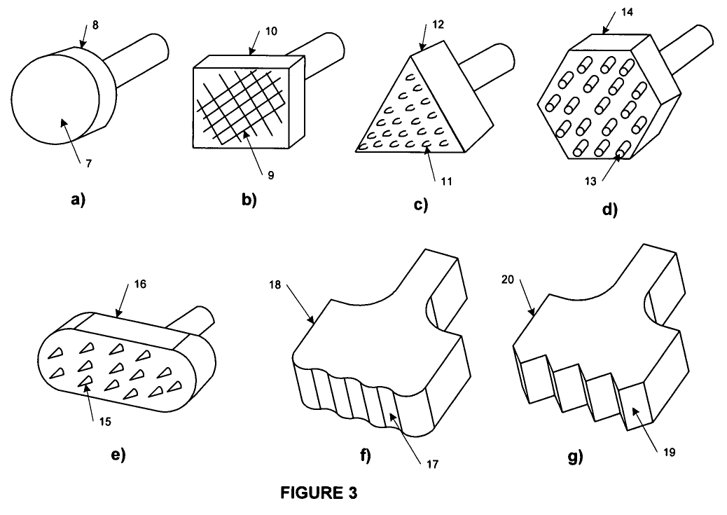

FIGS. 3a-3g are front-views of ultrasound tips that can be used with the ultrasound apparatus depicted in FIG. 1. FIG. 3a is an ultrasound tip that has a smooth front surface 7 and a circular peripheral boundary 8. FIG. 3b is an ultrasound tip that has a knurled front surface 9 and a rectangular peripheral boundary 10. FIG. 3c is an ultrasound tip that has a pyramidal front surface 11 and a triangular peripheral boundary 12. FIG. 3d is an ultrasound tip that has a cylindrical front surface 13 and a polygonal peripheral boundary 14. FIG. 3e is an ultrasound tip that has a spiky front surface 15 and an elliptical peripheral boundary 16. FIG. 3f is an ultrasound tip that has a waved front surface 17 and a rectangular peripheral boundary 18. FIG. 3g is an ultrasound tip that has a grooved front surface 19 and a rectangular peripheral boundary 20. These are only examples of front surfaces and peripheral boundaries of ultrasound tips that can be used with the ultrasound apparatus according to the present invention. Other front surfaces and peripheral boundaries may be similarly effective. Furthermore, any front surface can be mixed and matched with any peripheral boundary.

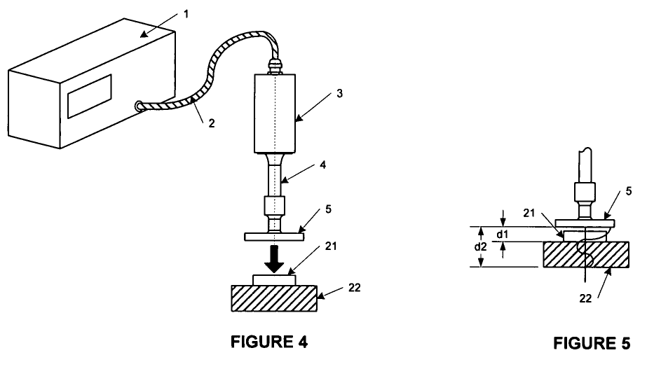

FIG. 4 is a perspective view of an ultrasound apparatus capable of energizing polymers through direct contact with a polymer. The ultrasound apparatus comprises an ultrasound power generator 1, a power supply cord 2, an ultrasonic transducer 3, an ultrasound horn 4, and an ultrasound tip 5. The ultrasound tip 5 delivers ultrasonic energy to the polymer 21 that is located on base material 22. Examples of polymer 21 to use include, but are not limited to, crystalline polymers, amorphous polymers, polymer alloys, or any other polymers currently approved for use in medical devices or food contact substances by the Federal Food and Drug Administration. Other polymers not currently approved may be similarly effective. The recommended polymer to use is a crystalline polymer. Examples of base material 22 on which to place the polymer 21 during delivery of ultrasonic waves include, but are not limited to, metals, polymers, elastomers, ceramics, rubbers, fabrics, composite materials, or any other similarly effective base materials or a combination thereof. An energized polymer 21 can be placed on a user to provide an analgesic effect.

FIG. 5 is a detailed view of the ultrasound apparatus depicted in FIG. 4 that can energize polymers through direct contact with a polymer. The ultrasound tip 5 delivers ultrasonic waves to the polymer 21 that is located on base material 22. Depending on the base material 22 used, ultrasound waves can travel through the base material 22 as shown in the sine wave that illustrates the emanated ultrasound energy. Depending upon the ultrasound parameters, reflection can occur both at the upper and lower surfaces levels of the base material 22; reflection of the ultrasonic waves can also occur at the lower surface level of the polymer 21. Finally, the amount of reflection may vary depending on the distance dl between the ultrasound tip 5 and the lower surface level of polymer 21 and may also vary depending on the distance d2 between the ultrasound tip 5 and the lower surface level of the base material 22. Reflection of ultrasonic waves can result in a polymer being double exposed to ultrasonic waves capable of energizing the polymer.

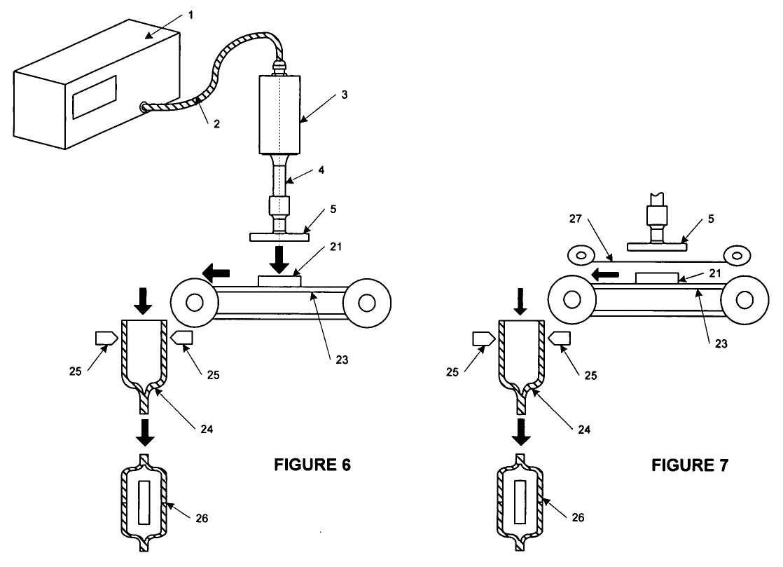

FIG. 6 is a perspective schematic view of a production line with an ultrasound apparatus capable of energizing polymers through direct contact. The ultrasound apparatus comprises an ultrasound power generator 1, a power supply cord 2, an ultrasound transducer 3, an ultrasound horn 4, and an ultrasound tip 5. The ultrasound tip 5 delivers ultrasonic waves to the polymer 21 that is located on base material 23. After being energized by exposure to ultrasonic waves, the polymer 21 moves down the production line and into storage material 24 that is secured by sealers 25, resulting in a sealed packet 26. Examples of storage material 24 to use include, but are not limited to, plastic bags, plastic sleeves, film, or fabric. Other storage materials may be similarly effective. The energized polymer 21 can be placed on a user to provide an analgesic effect. The use of the storage material 24 allows the polymer 21 to store energy, thus allowing the polymer 21 to be removed from the sealed packet 26 at a future time to be placed on a user provide an analgesic effect.

FIG. 7 is a perspective schematic view of a production line with an ultrasound apparatus capable of energizing polymers through a coupling medium. The ultrasound tip 5 delivers ultrasonic energy though a coupling medium 27 to the polymer 21 that is located on base material 23. Examples of coupling medium 27 include, but are not limited to, film, liquid, gel, or ointment. Other coupling mediums can be similarly effective. After being energized by exposure to ultrasonic waves, the polymer 21 moves down the production line and into storage material 24 that is secured by sealers 25, resulting in a sealed packet 26. Examples of storage material 24 to use include, but are not limited to, plastic bags, plastic sleeves, film, or fabric. Other storage materials may be similarly effective. The energized polymer 21 can be placed on a user to provide an analgesic effect. The use of the storage material 24 allows the polymer 21 to store energy, thus allowing the polymer 21 to be removed from the sealed packet 26 at a future time to be placed on a user provide an analgesic effect.

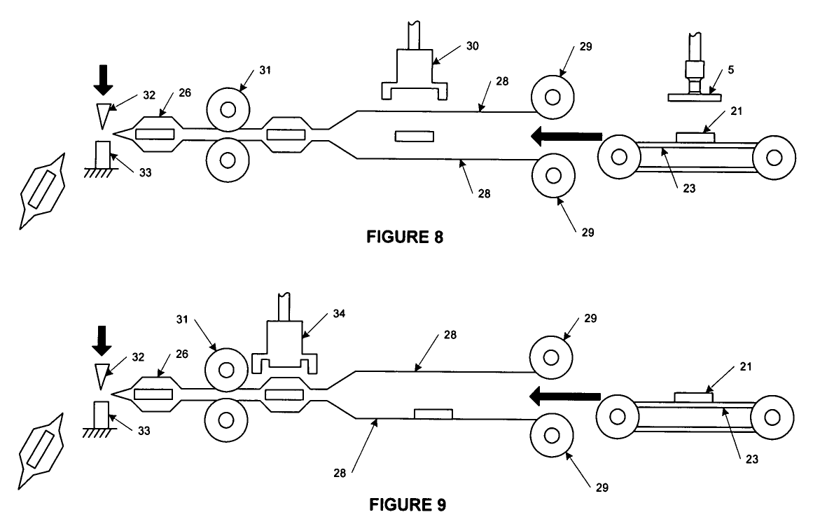

FIG. 8 is a perspective schematic view of a production line with an ultrasound apparatus capable of energizing polymers and with a separate device to seal polymers in storage. The ultrasound horn 5 delivers ultrasonic waves to the polymer 21 that is located on base material 23. After being energized by exposure to ultrasonic waves, the polymer 21 moves down the production line and into storage material 28 that is released from storage material spools 29. The storage material 28 may consist of one adhesive and one non-adhesive side, or it may also consist of two non-adhesive sides. Examples of storage material 28 to use include, but are not limited to, plastic bags, plastic sleeves, film, or fabric. Other storage materials may be similarly effective. The polymer 21 is then sealed in the storage material 28 by ultrasonic welding with ultrasound waves delivered from ultrasound tip 30. Ultrasound welding is an example of a sealing method; other methods, such as heat, may be similarly effective. The sealed packet 26 moves down the production line by driving wheels 31 where it is cut into an individual section by blade 32 contacting cutting block 33. Other methods and devices may be similarly effective in separating the sealed packet 26. The energized polymer 21 can be placed on a user to provide an analgesic effect. The use of the storage material 28 allows the polymer 21 to store energy, thus allowing the polymer 21 to be removed from the sealed packet 26 at a future time to be placed on a user to provide an analgesic effect.

FIG. 9 is a perspective view of a production line with an ultrasound apparatus capable of both energizing polymers and sealing the energized polymers in storage. Polymer 21 moves down production line into storage material 28 that is released from storage material spools 29. The ultrasound tip 34 then serves a dual function: the tip 34 delivers ultrasonic waves to the polymer 21 that is in the storage material 28, and the tip 34 also delivers ultrasonic waves to the storage material 28 in order to seal the polymer 21 in the storage material 28. Energizing the polymer 21 can occur before, during, or after ultrasound energy is delivered to seal the polymer 21 in the storage material 28. The sealed packet 26 moves down the production line by driving wheels 31 and then is cut into an individual section by blade 32 contacting cutting block 33. Other methods and devices may be similarly effective in separating the sealed packet 26. The energized polymer 21 can be placed on a user to provide an analgesic effect. The use of the storage material 28 allows the polymer 21 to store energy, thus allowing the polymer 21 to be removed from the sealed packet 26 at a future time to be placed on a user to provide an analgesic effect.

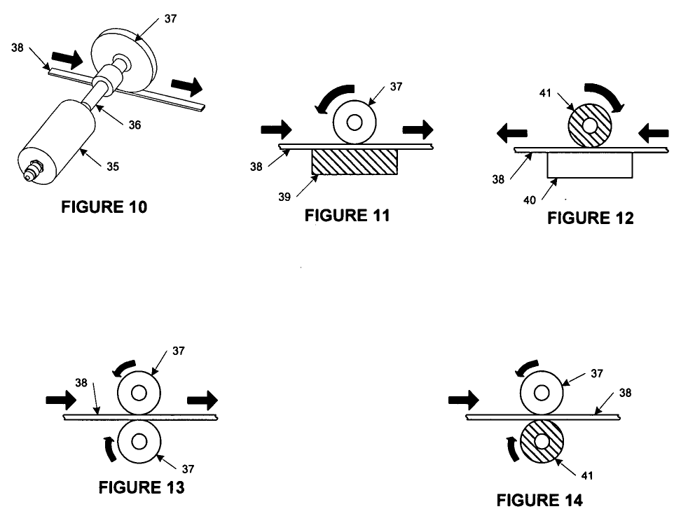

FIG. 10 is a perspective view of a production line with a rotating ultrasound apparatus that can energize moving polymers from the radial side of an ultrasound tip. The ultrasound apparatus consists of an ultrasonic transducer 35 that is connected to the ultrasound horn 36, and the ultrasound horn 36 is connected to the ultrasound tip 37. The ultrasound apparatus rotates and energizes the polymer 38 from the radial side of the ultrasound tip 37 as the polymer 38 moves down the production line. The recommended peripheral boundary for an ultrasound tip 37 on a rotating ultrasound apparatus is circular. Other peripheral boundaries may be similarly effective. The recommended radial surface for the ultrasound tip 37 is smooth. Other radial surfaces such as knurled, waved, or grooved (not shown) can be similarly effective. This production line method allows for large sections of polymer to be sonicated at once because after the moving polymer 38 has been energized, it can be cut into individual sections and sealed for use at a future time.

FIG. 11 is a cross-sectional view of a production line with a rotating ultrasound tip capable of energizing moving polymers from the radial side of the ultrasound tip. The moving polymer 38 moves down the production line to be energized by ultrasonic waves delivered from the radial side of the rotating ultrasound tip 37. There is base material 39 that is located in a fixed position on the other side of the moving polymer 38 from the rotating ultrasound tip 37. Once the moving polymer 38 has been energized, it can be cut into individual sections and sealed for use at a future time.

FIG. 12 is a cross-sectional view of a production line with an ultrasound tip in a fixed position that can energize moving polymers. The moving polymer 38 moves down production line to be energized by ultrasonic waves delivered from the radial side or distal end of the ultrasound tip 40 that is located in a fixed position. There is base material 41 located on the other side of the moving polymer 38 from the fixed ultrasound tip 40. The base material 41 rotates as the polymer 38 moves down the production line. Once the moving polymer 38 has been energized, it can be cut into individual sections and sealed for use at a future time.

FIG. 13 is a cross-sectional view of a production line with two rotating ultrasound tips capable of energizing moving polymers from the radial side of ultrasound tips. The moving polymer 38 moves down production line to be energized on each side by ultrasonic waves delivered from the radial sides of the rotating ultrasound tips 37. There no is base material in this production line. Once the moving polymer 38 has been energized, it can be cut into individual sections and sealed for use at a future time.

FIG. 14 is a cross-sectional view of a production line with a rotating ultrasound tip that is capable of energizing moving polymers from the radial side of the ultrasound tip. The moving polymer 38 moves down the production line to be energized by ultrasonic waves delivered from the radial side of the rotating ultrasound tip 37. There is base material 41 located on the other side of the moving polymer 38 from the rotating ultrasound tip 37. The base material 41 also rotates as the polymer 38 moves down the production line. Once the moving polymer 38 has been energized, it can be cut into individual sections and sealed for use at a future time.

The frequency range for the ultrasonic waves capable of energizing a polymer is approximately 15 kHz to approximately 40 MHz, with a preferred frequency range of approximately 20 kHz-approximately 40 kHz. The recommended low-frequency ultrasound value is approximately 30 kHz and the recommended high-frequency ultrasound value is approximately 3 MHz. The amplitude of the ultrasound waves can be 1 micron and above. The preferred amplitude range for low-frequency ultrasound is approximately 50 microns to approximately 60 microns, and the recommended amplitude value for low-frequency ultrasound is approximately 50 microns. The preferred amplitude range for high-frequency ultrasound is approximately 3 microns to approximately 10 microns, and the recommended amplitude value for high-frequency ultrasound is approximately 3 microns. The time of sonication will vary based on factors such as the ultrasound frequency, amplitude, intensity, the type of polymer, the thickness of polymer, the type of base material, the thickness of base material, etc.

Ultrasonic waves are delivered from an ultrasound apparatus to a polymer to energize the polymer. Ultrasonic waves can be delivered by either direct contact, through a coupling medium, or without contact. Ultrasonic waves can also be delivered from either the distal end or the radial side of the ultrasound horn/tip. The shape of the ultrasound tip used may vary. The peripheral boundary may be circular, rectangular, triangular, polygonal, elliptical, or another similar shape or combination of shapes. The front surface of the ultrasound tip may be smooth, knurled, pyramidal, cylindrical, spiky, waved, grooved or another similar surface or combination of surfaces. The preferred shape of the ultrasound tip is a smooth front surface with a rectangular peripheral boundary, but other shapes can also be similarly effective.

The polymer may be placed on surface material while being energized by exposure to ultrasonic waves. The surface materials that may be used vary from metals, polymers, elastomers, ceramics, rubbers, fabrics, composite materials, or any other similarly effective surface materials or a combination thereof. The size and thickness of the surface material can also vary. Besides acting as a base while the polymer is being energized, the surface material can also serve an additional purpose. Depending upon the surface material used and the parameters of the ultrasound waves delivered, ultrasound waves can reflect off of the surface material and back onto the polymer once again, thus resulting in the polymer being double exposed to ultrasonic waves capable of energizing the polymer. The ultrasonic waves can also reflect off the lower surface level of the polymer itself. The polymer can also be energized by means other than ultrasound such as UV, microwave, laser, electricity, RF, sun, light, magnetic/electromagnetic, etc.

The polymer may be placed in storage material before, after, or while being energized by ultrasonic waves. The polymer can be energized and then dropped into storage material, fed into storage material, or any other method to store an energized polymer. The polymer can also be fed into storage material so that it can energized and sealed simultaneously. Finally, the polymer can be sealed in its storage material and then it can be energized through the storage material.

The energized polymer can be placed on a user to provide an analgesic effect. The energized polymer can be removed from the storage material at a future to be placed on a user to provide an analgesic effect. The recommended use of the energized polymer is to place the energized polymer directly on the user's skin, and preferably to place the energized polymer on the user's pain area.

Although specific embodiments and methods of use have been illustrated and described herein, it will be appreciated by those of ordinary skill in the art that any arrangement that is calculated to achieve the same purpose may be substituted for the specific embodiments and methods shown. It is to be understood that the above description is intended to be illustrative and not restrictive. Combinations of the above embodiments and other embodiments as well as combinations of the above methods of use and other methods of use will be apparent to those having skill in the art upon review of the present disclosure. The scope of the present invention should be determined with reference to the appended claims, along with the full scope of equivalents to which such claims are entitled.

### REFERENCE LIST

### U.S. Patent Documents

U.S. Pat. No. 4,023,579 May, 1977 Suroff
U.S. Pat. No. 4,325,258 April, 1982 Foster
U.S. Pat. No. 4,325,381 April, 1982 Glenn
U.S. Pat. No. 4,339,952 July, 1982 Foster
U.S. Pat. No. 4,346,599 August, 1982 McLaughlin, et al.
U.S. Pat. No. 4,437,332 March, 1984 Pittaro
U.S. Pat. No. 4,488,000 December, 1984 Glenn
U.S. Pat. No. 4,507,582 March, 1985 Glenn
U.S. Pat. No. 4,532,938 August, 1985 Carlisle
U.S. Pat. No. 4,564,928 January, 1986 Glenn, et al.
U.S. Pat. No. 4,763,671 August, 1988 Goffinet
U.S. Pat. No. 4,763,677 August, 1988 Miller
U.S. Pat. No. 4,798,232 January, 1989 Stembridge, et al.
U.S. Pat. No. 4,834,102 May, 1989 Schwarzchild, et al.
U.S. Pat. No. 4,917,097 April, 1990 Proudian, deceased, et al.
U.S. Pat. No. 4,951,677 August, 1990 Crowley, et al.
U.S. Pat. No. 4,977,601 December, 1990 Bicz
U.S. Pat. No. 4,977,898 December, 1990 Schwarzschild, et al.
U.S. Pat. No. 5,115,805 May, 1992 Bommannan et al.
U.S. Pat. No. 5,141,677 August, 1992 Fogarty
U.S. Pat. No. 5,164,707 November, 1992 Rasmussen, et al.
U.S. Pat. No. 5,186,181 February, 1993 Franconi et al.
U.S. Pat. No. 5,231,975 August, 1993 Bommannan et al.
U.S. Pat. No. 5,323,769 June, 1994 Bommannan et al.
U.S. Pat. No. 5,334,181 August, 1994 Rubinsky et al.
U.S. Pat. No. 5,353,350 October, 1994 Unsworth, et al.
U.S. Pat. No. 5,406,951 April, 1995 Hoff, et al.
U.S. Pat. No. 5,413,550 May, 1995 Castel
U.S. Pat. No. 5,433,739 July, 1995 Sluijter et al.
U.S. Pat. No. 5,441,490 August, 1995 Svedman
U.S. Pat. No. 5,445,611 August, 1995 Eppstein et al.
U.S. Pat. No. 5,456,259 October, 1995 Barlow, et al.
U.S. Pat. No. 5,460,595 October, 1995 Hall et al.
U.S. Pat. No. 5,494,039 February, 1996 Onik et al.
U.S. Pat. No. 5,562,096 October, 1996 Hossack, et al.
U.S. Pat. No. 5,571,147 November, 1996 Sluijter et al.
U.S. Pat. No. 5,599,396 February, 1997 Sandhu
U.S. Pat. No. 5,633,584 May, 1997 Maryanskl, et al.
U.S. Pat. No. 5,654,279 August, 1997 Rubinsky et al.
U.S. Pat. No. 5,665,382 September, 1997 Grinstaff et al.
U.S. Pat. No. 5,674,218 October, 1997 Rubinsky et al.
U.S. Pat. No. 5,690,608 November, 1997 Watanabe, et al.
U.S. Pat. No. 5,709,855 January, 1998 Bockow
U.S. Pat. No. 5,720,743 February, 1998 Bischof et al.
U.S. Pat. No. 5,733,280 March, 1998 Avitall
U.S. Pat. No. 5,741,248 April, 1998 Stern et al.
U.S. Pat. No. 5,755,755 May, 1998 Panyard
U.S. Pat. No. 5,786,578 July, 1998 Christy et al.
U.S. Pat. No. 5,820,626 October, 1998 Baumgardner
U.S. Pat. No. 5,840,715 November, 1998 Florio
U.S. Pat. No. 5,853,290 December, 1998 Winston
U.S. Pat. No. 5,855,706 January, 1999 Grewell
U.S. Pat. No. 5,885,274 March, 1999 Fullmer et al.
U.S. Pat. No. 5,897,786 April, 1999 Henkel, et al.
U.S. Pat. No. 5,906,612 May, 1999 Chinn
U.S. Pat. No. 5,968,034 October, 1999 Fullmer et al.
U.S. Pat. No. 5,976,092 November, 1999 Chinn
U.S. Pat. No. 5,976,123 November, 1999 Baumgardner et al.
U.S. Pat. No. 6,007,570 December, 1999 Sharkey et al.
U.S. Pat. No. 6,011,022 January, 2000 El Khoury
U.S. Pat. No. 6,030,374 February, 2000 McDaniel
U.S. Pat. No. 6,048,337 April, 2000 Svedman
U.S. Pat. No. 6,058,938 May, 2000 Chu et al.
U.S. Pat. No. 6,063,108 May, 2000 Salansky et al.
U.S. Pat. No. 6,068,628 May, 2000 Fanton et al.
U.S. Pat. No. 6,073,051 Jun., 2000 Sharkey et al.
U.S. Pat. No. 6,095,149 August, 2000 Sharkey et al.
U.S. Pat. No. 6,126,682 October, 2000 Sharkey et al.
U.S. Pat. No. 6,136,795 October, 2000 Florio
U.S. Pat. No. 6,143,278 November, 2000 Elkhoury
U.S. Pat. No. 6,168,593 January, 2001 Sharkey et al.
U.S. Pat. No. 6,183,434 February, 2001 Eppstein
U.S. Pat. No. 6,200,308 March, 2001 Pope et al.
U.S. Pat. No. 6,210,393 April, 2001 Brisken
U.S. Pat. No. 6,231,528 May, 2001 Kaufman et al.
U.S. Pat. No. 6,245,347 June, 2001 Zhang et al.
U.S. Pat. No. 6,261,311 July, 2001 Sharkey et al.
U.S. Pat. No. 6,267,737 July, 2001 Meilus
U.S. Pat. No. 6,303,142 October, 2001 Zhang et al.
U.S. Pat. No. 6,306,431 October, 2001 Zhang et al.
U.S. Pat. No. RE37,463 December, 2001 Altman
U.S. Pat. No. 6,340,472 January, 2002 Zhang et al.
U.S. Pat. No. 6,391,026 May, 2002 Hung et al.
U.S. Pat. No. 6,398,753 June, 2002 McDaniel
U.S. Pat. No. 6,402,739 June, 2002 Neev
U.S. Pat. No. 6,410,599 June, 2002 Johnson
U.S. Pat. No. 6,413,253 July, 2002 Koop et al.
U.S. Pat. No. 6,414,032 July, 2002 Johnson
U.S. Pat. No. 6,416,705 July, 2002 Dinzburg, et al.
U.S Pat. No. 6,432,068 August, 2002 Corl, et al.
U.S. Pat. No. 6,432,102 August, 2002 Joye et al.
U.S. Pat. No. 6,436,078 August, 2002 Svedman
U.S. Pat. No. 6,443,898 September, 2002 Unger, et al.
U.S. Pat. No. 6,461,316 October, 2002 Lee et al.
U.S. Pat. No. 6,465,006 October, 2002 Zhang et al.
U.S. Pat. No. 6,478,754 November, 2002 Babaev
U.S. Pat. No. 6,478,793 November, 2002 Cosman et al.
U.S. Pat. No. 6,494,874 December, 2002 Brisken
U.S. Pat. No. 6,494,900 December, 2002 Salansky et al.
U.S. Pat. No. 6,503,243 January, 2003 Brisken
U.S. Pat. No. 6,514,244 February, 2003 Pope et al.
U.S. Pat. No. 6,517,536 February, 2003 Hooven et al.
U.S. Pat. No. 6,517,568 February, 2003 Sharkey et al.
U.S. Pat. No. 6,519,500 February, 2003 White
U.S. Pat. No. 6,533,803 March, 2003 Babaev
U.S. Pat. No. 6,544,401 April, 2003 Colic
U.S. Pat. No. 6,546,935 April, 2003 Hooven
U.S. Pat. No. 6,547,784 April, 2003 Thompson et al.
U.S. Pat. No. 6,547,810 April, 2003 Sharkey et al.
U.S. Pat. No. 6,562,033 May, 2003 Shah et al.
U.S. Pat. No. 6,569,099 May, 2003 Babaev
U.S. Pat. No. 6,601,581 August, 2003 Babaev
U.S. Pat. No. 6,613,044 September, 2003 Carl
U.S. Pat. No. 6,613,350 September, 2003 Zhang et al.
U.S. Pat. No. 6,622,731 September, 2003 Daniel et al.
U.S. Pat. No. 6,623,444 September, 2003 Babaev
U.S. Pat. No. 6,626,049 September, 2003 Ao
U.S. Pat. No. 6,638,276 October, 2003 Sharkey et al.
U.S. Pat. No. 6,639,872 October, 2003 Rein
U.S. Pat. No. 6,645,202 November, 2003 Pless et al.
U.S. Pat. No. 6,645,203 November, 2003 Sharkey et al.
U.S. Pat. No. 6,645,330 November, 2003 Pargass, et al.
U.S. Pat. No. 6,652,473 November, 2003 Kaufman et al.
U.S. Pat. No. 6,652,864 November, 2003 Webb et al.
U.S. Pat. No. 6,663,554 December, 2003 Babaev
U.S. Pat. No. 6,663,622 December, 2003 Foley et al.
U.S. Pat. No. 6,685,702 February, 2004 Quijano et al.
U.S. Pat. No. 6,712,816 March, 2004 Hung et al.
U.S. Pat. No. 6,723,064 April, 2004 Babaev
U.S. Pat. No. 6,726,685 April, 2004 To et al.
U.S. Pat. No. 6,733,496 May, 2004 Sharkey et al.
U.S. Pat. No. 6,736,835 May, 2004 Pellegrino et al.
U.S. Pat. No. 6,746,401 June, 2004 Panescu
U.S. Pat. No. 6,749,605 June, 2004 Ashley et al.
U.S. Pat. No. 6,752,805 June, 2004 Maguire et al.
U.S. Pat. No. 6,756,053 June, 2004 Zhang et al.
U.S. Pat. No. 6,759,434 July, 2004 Johnson
U.S. Pat. No. 6,761,715 July, 2004 Carroll
U.S. Pat. No. 6,761,729 July, 2004 Babaev
U.S. Pat. No. 6,767,347 July, 2004 Sharkey et al.
U.S. Pat. No. 6,780,426 August, 2004 Zhang et al.
U.S. Pat. No. 6,787,149 September, 2004 El Khoury et al.
U.S. Pat. No. 6,805,128 October, 2004 Pless et al.
U.S. Pat. No. 6,805,129 October, 2004 Pless et al.
U.S. Pat. No. 6,815,694 November, 2004 Sfez et al.
U.S. Pat. No. 6,832,997 December, 2004 Uchida et al.
U.S. Pat. No. 6,843,771 January, 2005 Lo, et al.
U.S. Pat. No. 6,855,133 February, 2005 Svedman
U.S. Pat. No. 6,868,286 March, 2005 Hille et al.
U.S. Pat. No. 6,878,155 April, 2005 Sharkey et al.
U.S. Pat. No. 6,881,214 April, 2005 Cosman et al.
U.S. Pat. No. 6,887,861 May, 2005 Hill et al.
U.S. Pat. No. 6,889,694 May, 2005 Hooven
U.S. Pat. No. 6,896,673 May, 2005 Hooven
U.S. Pat. No. 6,908,448 June, 2005 Redding, Jr.
U.S. Pat. No. 6,913,763 July, 2005 Lerner
U.S. Pat. No. 6,923,806 August, 2005 Hooven et al.
U.S. Pat. No. 6,932,811 August, 2005 Hooven et al.
U.S. Pat. No. 6,937,893 August, 2005 Danz et al.
U.S. Pat. No. 6,939,348 September, 2005 Malecki et al.
U.S. Pat. No. 6,974,454 December, 2005 Hooven
U.S. Pat. No. 6,984,233 January, 2006 Hooven
U.S. Pat. No. 6,997,941 February, 2006 Sharkey et al.
U.S. Pat. No. 7,001,415 February, 2006 Hooven
U.S. Pat. No. 7,004,933 February, 2006 McDaniel

I claim:

1) A method for creating ultrasound energized polymers, comprising the steps of:
a) delivering ultrasonic waves to a polymer; and
b) wherein the ultrasonic waves have an intensity capable of energizing a polymer.

2) The method according to claim 1, further comprising the steps of generating the ultrasonic waves with intensity capable of energizing a polymer.

3) The method according to claim 1, wherein the ultrasound frequency is in the range of approximately 15 kHz-approximately 40 MHz.

4) The method according to claim 1, wherein the preferred low-frequency ultrasound range is approximately 20 kHz-approximately 40 kHz.

5) The method according to claim 1, wherein the preferred high-frequency ultrasound range is approximately 1 MHz-approximately 5 MHz.

6) The method according to claim 1, wherein the recommended low-frequency ultrasound value is approximately 30 kHz.

7) The method according to claim 1, wherein the recommended high-frequency ultrasound value is approximately 3 MHz.

8) The method according to claim 1, wherein the ultrasound amplitude is at least 1 micron.

9) The method according to claim 1, wherein the preferred amplitude range for low-frequency ultrasound is approximately 50 microns-approximately 60 microns.

10) The method according to claim 1, wherein the preferred amplitude range for high-frequency ultrasound is approximately 3 microns-approximately 10 microns.

11) The A method according to claim 1, wherein the recommended amplitude value for low-frequency ultrasound is approximately 50 microns.

12) The method according to claim 1, wherein the recommend amplitude value for high-frequency ultrasound is approximately 3 microns.

13) The method according to claim 1, wherein the ultrasound waves are delivered to a polymer through direct contact.

14) The method according to claim 1, wherein the ultrasound waves are delivered to a polymer through a coupling medium.

15) The method according to claim 1, wherein the ultrasound waves are delivered to a polymer without contacting the polymer.

16) The method according to claim 1, wherein the ultrasound waves are delivered to the polymer for at least of 0.1 seconds.

17) The method according to claim 1, wherein the recommended duration range to deliver low-frequency ultrasound waves is approximately 30 seconds to approximately 1 minute.

18) The method according to claim 1, wherein the recommended duration to deliver high-frequency ultrasound waves is approximately 3 minutes.

19) The method according to claim 1, wherein during delivery of the ultrasonic waves the polymer is placed on a base material such as a metal, polymer, elastomer, ceramic, rubber, fabric, composite material, or any other material or any combination thereof

20) The method according to claim 1, further comprising the step of storing the energized polymer such that the energy in said polymer does not wholly dissipate.

21) The method according to claim 20, wherein said storage means is a plastic bag.

22) The method according to claim 21, further comprising the step of sealing said plastic bag.

23) The method according to claim 20, wherein said storage means is a plastic sleeve.

24) The method according to claim 23, further comprising the step of sealing said plastic sleeve.

25) The method according to claim 20, wherein said storage means comprises:
a) two pieces of film; and
b) a means of detachably adhering said films to said energized polymer;
wherein one piece of said film is adhered by said means to one surface of said energized polymer and the other piece of said film is adhered by said means to the opposite surface of said energized polymer.

26) The method according to claim 20, wherein said storage means comprises:
a) two pieces of film;
b) a means of detachably adhering one piece of said film to said energized polymer; and
c) a means of permanently adhering one piece of said film to said energized polymer;
wherein one piece of said film is permanently adhered by said means to one surface of said energized polymer and the other piece of said film is detachably adhered by said means to the opposite surface said energized polymer.

27) The method according to claim 1, wherein the polymer consists of a material such as crystalline polymer, amorphous polymer, polymer alloy, polymers approved for use in medical devices or food contact substances by the Federal Food and Drug Administration, or other polymers not currently approved.

28) The method according to claim 1, wherein polymers move down a production line system to be directly energized by an ultrasound apparatus/system and then placed in a means of storage.

29) The method according to claim 1, wherein polymers move down a production line to be energized through a coupling medium by an ultrasound apparatus/system and then placed in a means of storage.

30) The method according to claim 1, wherein polymers move down a production line to be energized by an ultrasound apparatus/system and then sealed in a means of storage by a separate apparatus/system.

31) The method according to claim 1, wherein polymers move down a production line to be energized and sealed in a means of storage by the same apparatus/system.

32) A method for pain relief using energized polymers, wherein the energized polymer is placed on a user to provide an analgesic effect.

33) The method according to claim 32, wherein the polymer is placed on the user's skin.

34) The method according to claim 32, wherein the polymer is placed on the user's pain area.

35) The method according to claim 32, wherein the polymer is placed on the user immediately after being energized.

36) The method according to claim 32, further comprising the step of removing said energized polymer from storage means before placing on a user.

37) The method according to claim 32, wherein the polymer is energized by ultrasound energy.

38) The method according to claim 32, wherein the polymer is energized by energy such as UV, microwave, laser, electricity, RF, sun, light, magnetic/electromagnetic, etc.

39) An apparatus for creating ultrasound energized polymers, comprising:
a) an ultrasound apparatus/system for generating ultrasonic waves;
b) wherein the apparatus/system delivers ultrasonic waves to a polymer; and
c) wherein the ultrasonic waves have an intensity capable of energizing a polymer.

40) The apparatus according to claim 39, wherein the ultrasound apparatus/system generates the ultrasonic waves with particular ultrasound parameters indicative of an intensity capable of energizing the polymer.

41) The apparatus according to claim 39, wherein the ultrasound frequency is in the range of approximately 15 kHz-approximately 40 MHz.

42) The apparatus according to claim 39, wherein the preferred low-frequency ultrasound range is approximately 20 kHz-approximately 40 kHz.

43) The apparatus according to claim 39, wherein the preferred high-frequency ultrasound range is approximately 1 MHz-approximately 5 MHz.

44) The apparatus according to claim 39, wherein the recommended low-frequency ultrasound value is approximately 30 kHz.

45) The apparatus according to claim 39, wherein the recommended high-frequency ultrasound value is approximately 3 MHz.

46) The apparatus according to claim 39, wherein the ultrasound amplitude is at least 1 micron.

47) The apparatus according to claim 39, wherein the preferred amplitude range for low-frequency ultrasound is approximately 50 microns-approximately 60 microns.

48) The apparatus according to claim 39, wherein the preferred amplitude range for high-frequency ultrasound is approximately 3 microns-approximately 10 microns.

49) The apparatus according to claim 39, wherein the recommended amplitude value for low-frequency ultrasound is approximately 50 microns.

50) The apparatus according to claim 39, wherein the recommend amplitude value for high-frequency ultrasound is approximately 3 microns.

51) The apparatus according to claim 39, further comprising a means for measuring the period of time during which ultrasonic waves are delivered to the polymer.

52) The apparatus according to claim 51, wherein said means for measuring time is a timer.

53) The apparatus according to claim 39, wherein ultrasonic waves are delivered to the polymer for a duration of at least 0.1 seconds.

54) The apparatus according to claim 39, wherein the ultrasonic waves are delivered to a polymer through direct contact.

55) The apparatus according to claim 39, wherein the ultrasonic waves are delivered to a polymer through a coupling medium.

56) The apparatus according to claim 39, wherein the ultrasonic waves are delivered to a polymer without contacting the polymer.

57) The apparatus according to claim 39, wherein during delivery of the ultrasonic waves the polymer is placed on a base material such as a metal, polymer, elastomer, ceramic, rubber, fabric, composite material, or any other material or any combination thereof.

58) The apparatus according to claim 39, wherein the energized polymer is placed in storage or sealed to be used at a later time.

59) The apparatus according to claim 39, further comprised of a means for storing the energized polymer such that the energy in said polymer does not wholly dissipate.

60) The apparatus according to claim 59, wherein said storage means is a plastic bag.

61) The apparatus according to claim 60, further comprised of a means for sealing said plastic bag.

62) The apparatus according to claim 59, wherein said storage means is a plastic sleeve.

63) The apparatus according to claim 62, further comprised of a means for sealing said plastic sleeve.

64) The apparatus according to claim 59, wherein said storage means comprises:
a) two pieces of film; and
b) a means of detachably adhering said films to said energized polymer;
wherein one piece of said film is adhered by said means to one surface of said energized polymer and the other piece of said film is adhered by said means to the opposite surface of said energized polymer.

65) The apparatus according to claim 59, wherein said storage means comprises:
a) two pieces of film;
b) a means of detachably adhering one piece of said film to said energized polymer; and
c) a means of permanently adhering one piece of said film to said energized polymer;
wherein one piece of said film is permanently adhered by said means to one surface of said energized polymer and the other piece of said film is detachably adhered by said means to the opposite surface said energized polymer.

66) The apparatus according to claim 59, further comprising a means of sealing said storage means.

67) The apparatus according to claim 66, wherein the ultrasound apparatus both energizes the polymer and seals the polymer in storage.

68) The apparatus according to claim 39, wherein the transducer contains a radiation surface having a surface area dimensioned/constructed for achieving delivery of the ultrasonic waves to a polymer with an intensity capable of energizing the polymer.

69) The apparatus according to claim 39, where the surface area of the distal end of the radiation surface is flat, pyramidal, knurled, cylindrical, spiked, ruffled, grooved, or another comparable shape or combination of shapes.

70) The apparatus according to claim 39, wherein the surface area of the radial side of the radiation surface flat, ruffled, grooved, knurled, or another comparable shape or combination of shapes.

71) The apparatus according to claim 39, wherein the shape of the peripheral boundary of the radiation surface is intended to achieve delivery of the ultrasonic waves to the polymer with an intensity capable energizing the polymer.

72) The apparatus according to claim 39, wherein the shape of the peripheral boundary of the radiation surface is circular, elliptical, rectangular, polygonal, or another comparable shape or combination of shapes.

73) The apparatus according to claim 39, wherein the transducer is driven by a continuous or pulsed frequency.

74) The apparatus according to claim 39, wherein the transducer is driven by a fixed or modulated frequency.

75) The apparatus according to claim 39, wherein the driving wave form of the transducer is selected from the group consisting of sinusoidal, rectangular, trapezoidal and triangular wave forms.

76) An energized polymer, comprising:
a) a piece of polymer; and
b) wherein the polymer has been energized through the deliver of energy to the polymer.

77) The energized polymer according to claim 76, further comprising a means of storing the energized polymer such that the energy in said polymer does not wholly dissipate.

78) The energized polymer according to claim 77, wherein said storage means is a plastic bag.

79) The energized polymer according to claim 78, further comprising a means of sealing said plastic bag.

80) The energized polymer according to claim 77, wherein said storage means is a plastic sleeve.

81) The energized polymer according to claim 80, further comprising a means of sealing said plastic sleeve.

82) The energized polymer according to claim 77, wherein said storage means comprises:
a) two pieces of film; and
b) a means of detachably adhering said films to said energized polymer,
wherein one piece of said film is adhered by said means to one surface of said energized polymer and the other piece of said film is adhered by said means to the opposite surface of said energized polymer.

83) The energized polymer according to claim 77, wherein said storage means comprises:
a) two pieces of film;
b) a means of detachably adhering one piece of said film to said energized polymer; and
c) a means of permanently adhering one piece of said film to said energized polymer,
wherein one piece of said film is permanently adhered by said means to one surface of said energized polymer and the other piece of said film is detachably adhered by said means to the opposite surface said energized polymer.

84) The energized polymer according to claim 77, further comprising a means of sealing said storage means.

85) The energized polymer according to claim 76, wherein the energy delivered the polymer is ultrasonic energy.

86) The energized polymer according to claim 76, wherein the energy delivered to the polymer is energy such as UV, microwave, laser, electricity, RF, sun, light, magnetic/electromagnetic, etc.

87) The energized polymer according to claim 85, wherein the polymer is energized by delivering ultrasound with a frequency range of approximately 15 kHz to approximately 40 MHz.

88) The energized polymer according to claim 85, wherein the polymer is energized by delivering low-frequency ultrasound with a preferred frequency range of approximately 20 kHz to approximately 40 kHz.

89) The energized polymer according to claim 85, wherein the polymer is energized by delivering high-frequency ultrasound with a preferred frequency of range approximately 1 MHz to approximately 5 MHz.

90) The energized polymer according to claim 85, wherein the polymer is energized by delivering low-frequency ultrasound with a recommended frequency value of approximately 30 kHz.

91) The energized polymer according to claim 85, wherein the polymer is energized by delivering high-frequency ultrasound with a recommended frequency value of approximately 3 MHz.

92) The energized polymer according to claim 85, wherein the polymer is energized by delivering ultrasound with an amplitude of at least 1 micron.

93) The energized polymer according to claim 85, wherein the polymer is energized by delivering low-frequency ultrasound with a preferred amplitude range of approximately 50 to approximately 60 microns.

94) The energized polymer according to claim 85, wherein the polymer is energized by delivering high-frequency ultrasound with a preferred amplitude range of approximately 3 to approximately 10 microns.

95) The energized polymer according to claim 85, wherein the polymer is energized by delivering low-frequency ultrasound with a recommended amplitude value of approximately 50 microns.

96) The energized polymer according to claim 85, wherein the polymer is energized by delivering high-frequency ultrasound with a recommended amplitude value of approximately 3 microns.

97) The energized polymer according to claim 85, wherein the polymer is energized by delivering ultrasound waves to the polymer while the polymer is on a base material such as a metal, polymer, elastomer, ceramic, rubber, fabric, composite materials, or any other material or any combination thereof.

98) The energized polymer according to claim 76, wherein the energized polymer can provide an analgesic effect.
